(19) United States
(12) Patent Application Publication
(10) Pub. No.: US 2007/0265579 A1
(43) Pub. Date: Nov. 15, 2007

(54) Dental syringe

(76) Inventors: Gennady Kleyman, Brooklyn, NY (US); Alexander Merson, Brooklyn, NY (US)

Correspondence Address: DARBY & DARBY P.C., P.O. BOX 770, Church Street Station, New York, NY, 10008-0770, US

(21) Appl. No.: 11/432,687

(22) Filed: May 10, 2006

(51) Int. Cl. A61M 5/00

(52) U.S. Cl. 604/207

(57) ABSTRACT

According to one exemplary embodiment of the present invention, a dental syringe includes a barrel extending along a longitudinal axis and including an interior space and a longitudinal window formed in a side wall of the barrel for receiving a cartridge that contains medication. A flexible flange member is formed at a proximal end of the barrel with one end of the flexible flange member extending into an opening at the proximal end that forms an entrance into the interior space of the barrel. The dental syringe has a plunger received in and axially displaceable within the interior space, with the plunger having a plurality of spaced grooves provided on an outer surface thereof. The flange is configured to selectively engage the grooves while generating an indicating signal corresponding to a predetermined dosage of medication dispensed from the cartridge during axial displacement of the plunger as the flange engages successive grooves.

### TECHNICAL FIELD

The present invention relates to dispensing devices, and more particularly, the present invention relates to a dental syringe for administering a predetermined dosage of medication to a patient.

### DESCRIPTION OF THE RELATED ART

A conventional dental syringe is formed of a metal body and a metal plunger that is slidably received within a portion of the body for administering the medication. The medication is contained within a disposable cartridge that is inserted through an opening formed in the body and is held in place within the body below the plunger. Once the cartridge is contained and held within the body, the plunger engages the cartridge to lock it in place and advancement of the plunger in an axial direction causes a force to be applied to the cartridge to cause the medication to be discharged from the cartridge through the syringe body and needle.

However, due to the nature of dental injections, it is typically a difficult process to control the precise amount of medication that is dispensed by the dentist into the patient. The amount of medication that is dispensed by the dentist depends on the degree that the plunger is advanced within the body and the degree of force applied to the cartridge. Most dentists learn to make educated guesses as to the amount of medication being dispensed based on their experience over time and in particular, the dentist develops a feel as to how much medication is being dispensed based on the degree of travel of the plunger within the body. For example, the dentist learns over time that for an average size adult, advancement of the plunger a certain distance yields a proper dosage of medication being dispensed. For different sized patients, the plunger is advanced different distances. However, this manner of dispensing the medication has a high degree of imprecision since the dentist is judging the amount of the dose based on the degree that the plunger is advanced within the body. For example, if the dentist requires additional medication, the dentist simply advances the plunger further within the body and into greater contact with the cartridge to expel additional medication.

Unlike other types of syringes, dental syringes typically do not include a graduated scale disposed on the body of the syringe to assist individual administering of medication by advancing the plunger based on information obtained from the graduated scale. However, this common measurement procedure can often be difficult and time-consuming, and more importantly, can be quite wasteful, as a quantity of the medication is often discarded in order to achieve the appropriate dosage.

Quite often, medical professionals, such as dentists, administering an injection may not have a clear view of the targeted body part of the patient, e.g., a gum portion surrounding a target tooth. In this case, many rely on their experience to administer the desired dosage, which still may not be precise administering technique.

A need thus exists for a dental syringe that has a structure configured to reliably produce a distinct audible and tactile signal to the user indicating that the desired dosage of medication has been discharged from the cartridge in a reliable, simple fashion.

### SUMMARY

The present invention is directed to dental syringes that satisfy these needs. The invention includes a dental syringe capable of producing a clear indicating signal, such as sound and/or pointed impulse sensed by the user while dispensing medication from a cartridge.

According to one exemplary embodiment of the present invention, a dental syringe includes a barrel extending along a longitudinal axis and including an interior space and a longitudinal window formed in a side wall of the barrel for receiving a cartridge that contains medication. A flexible flange member is formed at a proximal end of the barrel with one end of the flexible flange member extending into an opening at the proximal end that forms an entrance into the interior space of the barrel.

The dental syringe has a plunger received in and axially displaceable within the interior space, with the plunger having a plurality of spaced grooves provided on an outer surface thereof. The flange is configured to selectively engage the grooves while generating an indicating signal corresponding to a predetermined dosage of medication dispensed from the cartridge during axial displacement of the plunger as the flange engages successive grooves.

In yet another embodiment, a dental syringe includes a barrel extending along a longitudinal axis and including an interior space and a longitudinal window formed in a side wall of the barrel for receiving a cartridge that contains medication. The dental syringe has a flexible flange member formed at a proximal end of the barrel with one end of the flexible flange member extending into an opening at the proximal end that forms an entrance into the interior space of the barrel.

A plunger is received in and axially displaceable within the interior space and the plunger has a first set of grooves formed therein along a longitudinal length of the plunger and spaced a first distance apart from one another and a second set of grooves formed therein along the longitudinal length of the plunger and spaced a second distance from one another that is different from the first distance. The flexible flange member is configured to selectively engage one of the first set of grooves and the second set of grooves while generating an indicating signal corresponding to a predetermined dosage of medication dispensed from the cartridge during axial displacement of the plunger as the flexible flange member engages successive grooves.

These and other features and aspects of the present invention will be better understood with reference to the following description, figures, and appended claims.

### BRIEF DESCRIPTION OF THE DRAWING FIGURES

The above and other aspects and advantages of the present invention will be more clearly understood from the following detailed description taken in conjunction with the accompanying drawings, in which:

FIG. 1 is a perspective, partially exploded, view of a dental syringe, according to a first embodiment, with a displaceable plunger and a cartridge containing medication that is illustrated prior to insertion into a body of the dental syringe;

FIG. 2 is a perspective view of the dental syringe of FIG. 1 with the cartridge being inserted into the syringe body;

FIG. 3 is an enlarged perspective view of a portion of the dental syringe taken along the circle III of FIG. 1 to illustrate a dosage dispensing feature according to a first embodiment;

FIG. 4 is an enlarged perspective view of a portion of the dental syringe to illustrate a dosage dispensing feature according to a second embodiment and illustrating in an engaged position;

FIG. 5 is an enlarged perspective view of the dental syringe of FIG. 4, with the dosage dispensing feature being in a disengaged position;

FIG. 6 is an enlarged perspective view of a portion of the dental syringe to illustrate a dosage dispensing feature according to a third embodiment and in a disengaged position;

FIG. 7 is an enlarged perspective view of the dental syringe of FIG. 6, with the dosage dispensing feature being in an engaged position;

FIG. 8 is a cross-sectional view of the dental syringe in the engaged position of FIG. 7;

FIG. 9 is a perspective view of a dental syringe according to another embodiment, with a displaceable plunger having first and second dosage dispensing features to selectively permit two different dosage amounts to be administered with the same plunger depending upon the orientation of the plunger;

FIG. 10 is another perspective view of the dental syringe of FIG. 9 showing the other dosage dispensing feature;

FIG. 11 is an enlarged local perspective view showing the plunger in a completely disengage state;

FIG. 12 is an enlarged local perspective view showing the one set of dosage dispensing features of the plunger in an engaged state; and

FIG. 13 is an enlarged local perspective view showing the other set of dosage dispensing features of the plunger in an engaged state.

### DETAILED DESCRIPTION OF PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, left, right, up, down, over, above, below, beneath, rear, and front may be used with respect to the drawings. These and similar directional terms should not be construed to limit the scope of the invention in any manner. While, the present invention is described in sections as relating to dental syringes, it will be understood that a number of features described herein can be incorporated into other syringe designs as described in more detail below.

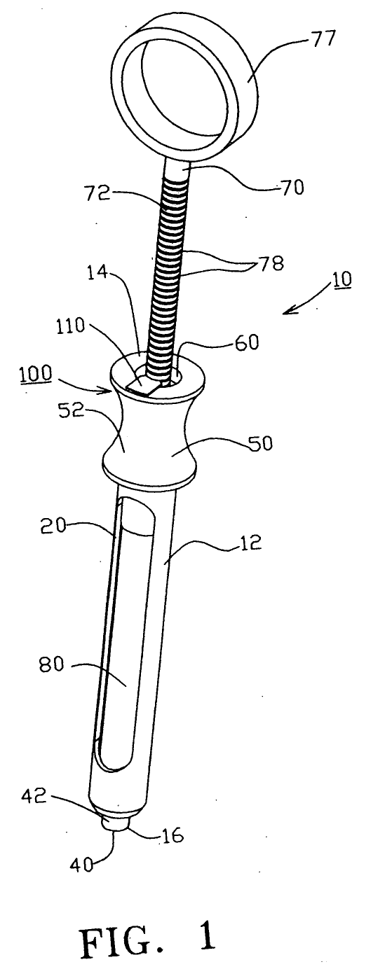

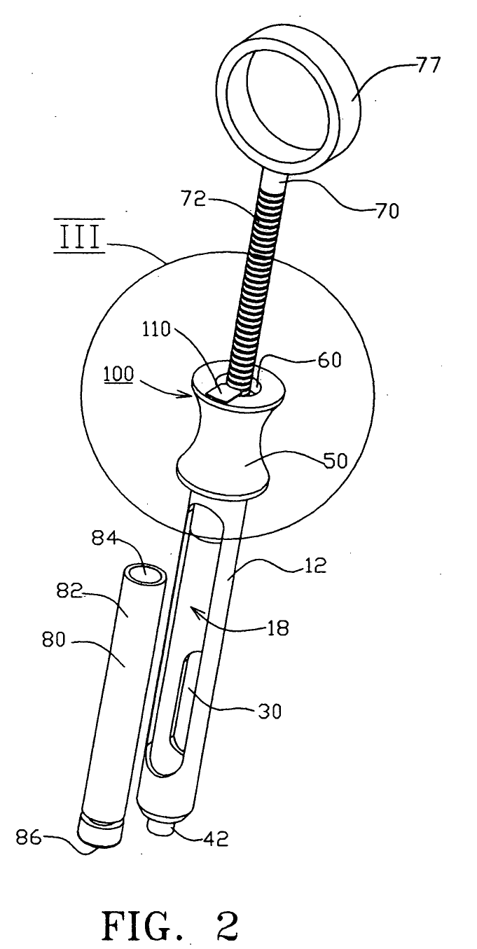

Referring more particularly to the drawings, FIGS. 1-3 illustrate a dosage dispensing device 10 including, but not limited to, a dental syringe or the like, which is operative to dispense predetermined dosages of medication. For purposes of illustration, the device 10 is described below and illustrated in the FIGS. 1-3 as being a dental syringe; however, it will be understood that this is not limiting of the scope of the present invention.

As described below in detail below, a clear sound signal accompanies each predetermined dosage dispensed from the dental syringe 10. Furthermore, the user experiences increased resistance during displacement of the components of the dental syringe indicated by a pointed impulse every time the predetermined dosage of medication has been displaced (discharged) similar to the feel experienced when operating a ratchet type tool.

The dental syringe 10 includes a hollow body or barrel 12 that has a proximal end 14 and an opposing distal end 16 through which the medication is dispensed. The dimensions and shape of the body 12 can vary depending upon the particular type of application; however, the body 12 is typically a cylindrically shaped metal body. The body 12 has a first opening or slot 20 and a second opening or slot 30, both of which are formed completely through the body 12 to provide access to an interior 18 of the body 12. Typically, the size (dimensions/area) of one of the openings 20, 30 is greater than the size of the other one and in particular, the first opening 20 has greater dimensions than the second opening 30. In the illustrated embodiment, the first opening 20 and the second opening 30 are each in the form of an elongated slot that extends along the longitudinal axis (length) of the body 12. The first opening 20 and second opening 30 are at least partially aligned along a transverse plane across the width of the body 12. The width of the first opening 20 is such that the opening 20 is formed in one-half of the body 12, while the second opening 30 is formed in the other half of the body 12; however, this is not critical but is merely exemplary of one embodiment.

The distal end 16 of the body 12 includes a dispensing opening 40 through which the medication is dispensed as described below. The dispensing opening 40 typically has dimensions (e.g., diameter) that are less than the dimensions (e.g., diameter) of the body 12 and in the illustrated embodiment, the distal end 16 of the body 12 tapers inwardly to a boss 42 that defines the opening 40. An annular shoulder is formed between the body 12 and the boss 42. Both the first and second openings 20, 30 do not extend to the location where the boss 42 is formed and projects from the body 12.

At the proximal end 14, a feature 50 is provided to assist the user in holding and manipulating the dental syringe 10. For example, the feature 50 can be in the form of a finger holder that is contoured and configured so that the user can easily grasp and hold the dental syringe 10 by means of the feature 50. The illustrated feature 50 is in the form of an enlarged body portion that generally has an hourglass shape defined by a smooth annular surface 52 of varying diameter so as to form the hourglass shape. One end of the feature 50 is positioned adjacent one end of the cylindrically shaped body 12 which has a diameter less than the diameter of the adjacent end of the feature 50.

Like the body 12, the boss 42 and the feature 50 are preferably formed of a metal material and along with the body 12 form an integral syringe structure. The boss 42 can also be configured to mate with a cannula, such as a sharp needle, (not shown) that is designed to be inserted into the tissue of the patient for administering the medication to the localized tissue area, such as the gum of a patient's mouth. For example, the boss 42 can include coupling features, such as threads, that are complementary to coupling features formed as part of the cannula to permit the cannula to be releasably attached to the boss 42. The cannula is a hollow member and when attached to the boss 42, the interior of the cannula is in fluid communication and axially aligned with the dispensing opening 40 so that the medication dispensed from the cartridge 80 is introduced into the interior of the cannula where it travels to the distal opening of the cannula and into the tissue.

At the proximal end 14, an opening 60 is provided and forms an entrance into the interior 18 of the body 12 to permit a plunger 70 to be received into and slideably moved within the interior 18 of the body 12, as well as be removed therefrom. The plunger 70 is similar to a standard dental syringe plunger in that it is defined by an elongated body (rod) 72 that has a proximal end 74 and an opposing distal end. At the proximal end 74, a handle or grasp 77 is formed to permit the user to hold and easily move the plunger in opposing axial directions (i.e., a push direction toward the body 12 and a pull direction away from the body 12). The handle 77 can be in the form of a ring-shaped structure (thumbring) that is formed at the end 74 and permits insertion of the user's finger or thumb to securely grasp and manipulate the plunger 70 (e.g., longitudinally slide the plunger 70 within the syringe body 12).

At the distal end of the plunger 70, a stopper or the like (not shown) with a pointed barb structure can be provided for causing the medication to be dispensed through the dispensing opening 40. The medication is contained within a disposable cartridge 80 that is shown in FIG. 1 prior to insertion into the body 12. The cartridge 80 is defined by a cartridge body 82 that has a first end 84 that faces the proximal end 14 of the body 12 and an opposing second end 86 that faces the distal end 16 of the body 12. The cartridge 80 has a shape that is complementary to the shape of the interior 18 since the cartridge 80 is inserted into and held within the interior 18 during operation of the dental syringe 10.

In the illustrated embodiment, the cartridge 80 is a cylindrically shaped canister that contains the medication and is sized so that the cartridge 80 can be inserted through the first opening 20 and into the interior 18. The cartridge 80 is thus inserted into and removed from the interior 18 by means of the first opening (slot) 20. Opposite the elongated slot 20 is the smaller slot 30 which serves as a barrel window for the purpose of the operator to see the cartridge 80 from the opposite side of the syringe during use. During syringe use the plunger rod 72 is initially retracted allowing for insertion of the cartridge 80 into the interior 18 of the body 12. The thumbring 77 is struck by hand to engage the barb into a rubber stopper or septum that seals the proximal end of the cartridge 80 and is easily rupturable under an applied force of the barb structure. The anterior wall of the cartridge 80 can be sealed by a cartridge cap that includes a membrane through which the medication is dispensed. The plunger 70 dispenses the medication by being further advanced longitudinally within the interior 18 and at the same time, the stopper/barb structure is longitudinally advanced within the interior of the cartridge 80 so as cause additional medication to be dispensed through the opposite end of the cartridge 80.

In accordance with the present invention, a dosage dispensing feature, generally indicated at 100, is provided to accurately dispense a predetermined dosage amount based on the controlled advancement of the plunger 70 within the interior 18 of the body 12. The dosage dispensing feature 100 according to a first embodiment is in the form of a flexible flange or finger 110 that extends at least partially into the opening 60 formed at the proximal end 14 of the syringe body 12. Thus, one end 112 of the flexible flange 110 is located within the opening 60, with the opposite end 114 being securely coupled to the body 12 to permit the flange 110 to flex (upwardly and downwardly) at the end 112. The illustrated flange 110 has a generally rectangular shape and is formed of a material that permits the flange 110 to flex under an applied force and then return to the rest position once the applied force is removed. For example, the flange 110 can be formed of a rigid plastic material or other material that offers the desired flexing action and in some embodiments, the flange 110 can be formed of a thin metal structure that can flex under applied force and return to the rest position.

The plunger 70 is constructed to mate with the dosage dispensing feature 100 and in particular, the plunger rod 72 has a plurality of recesses or channels or grooves 78 formed therein and along the longitudinal length of the plunger rod 72 that are configured to mate with the end 112 of the flange 110. The channels 78 are formed in a parallel spaced relationship at predetermined distances between adjacent channels 78. The distance between adjacent channels 78 defines the dosage amount that is dispensed upon one incremental longitudinal movement of the plunger 70 within the body 12. The plunger 70 is typically formed of a metal material and thus, the channels 78 can be machines into the plunger rod 72. It will also be appreciated that the channels 78 can be complete annular shaped grooves that extend around the rod 72 or the channels 78 can extend partially around the circumference of the rod 72 so long as the end 112 of the flange 110 can engage and be received in the channel 78.

As the plunger 70 moves relative to the flange 110, the flange 110 engages each of the plunger channels/recesses 78 to produce a sound signal. Consecutive sound signals and/or pointed impulses produced by the flexing of the flange 110 during displacement of the plunger 70 a predetermined distance indicate that a predetermined dosage of medication has exited the cartridge 80. In other words, the successive movement of the flange 110 from engagement with one channel or recess 78 to engagement with an adjacent channel or recess 78 due to the flexing action of the flange 110 results in a predetermined amount of medication being dispensed. It will be appreciated that the greater the distance between the two channels 78, the greater the amount of medication that is dispensed each incremental advancement of the plunger. In addition, the user may wish to longitudinally move the plunger 70 so that the flange 110 engages more than two successive channels or recesses 78. Each successive engagement of the flange 110 with one channel 78 results in another unit of medication being dispensed, with the understanding that for an adult sized dosage, the plunger 70 may need to be advanced so that the flange 110 successively engages a plurality of recesses 78.

It will also be appreciated that the use of different plungers 70 with the body 12 can result in different dosage amounts being dispensed since the spacing of the recesses 78 can be varied for each plunger 70, thereby directly influencing the amount of medication that is dispensed upon one “click” of the plunger 70. The advancement of the plunger 70 resembles a ratcheting action where the flange successively engages grooves formed along the length of the plunger to cause incremental advancement of the plunger.

FIG. 3 illustrates the engagement between the end 112 of the flange 110 and one channel or recess 78. It will also be appreciated that when the end 112 of the flange 110 engages one channel or recess 78, the plunger 70 is securely held in place and can not freely move in a longitudinal direction within the body 112. However, the flexibility and memory characteristics of the flange 110 permit the plunger 70 to advance within the body 12 once a sufficient force is applied to the plunger 70 to cause the flange 110 to disengage the channel 78 and as soon as the plunger 70 moves one increment and the next adjacent channel 78 aligns with the end 112, the flange 110 is biased back into engagement with this next adjacent channel 78.

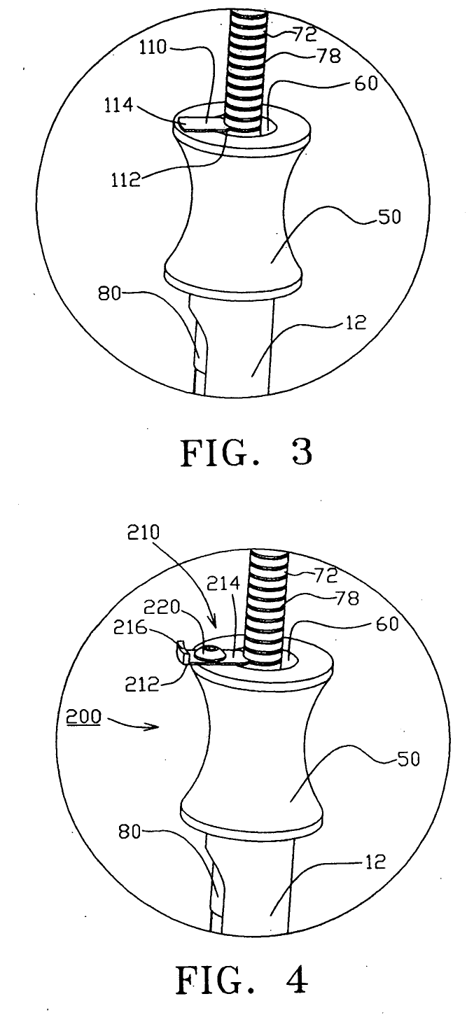

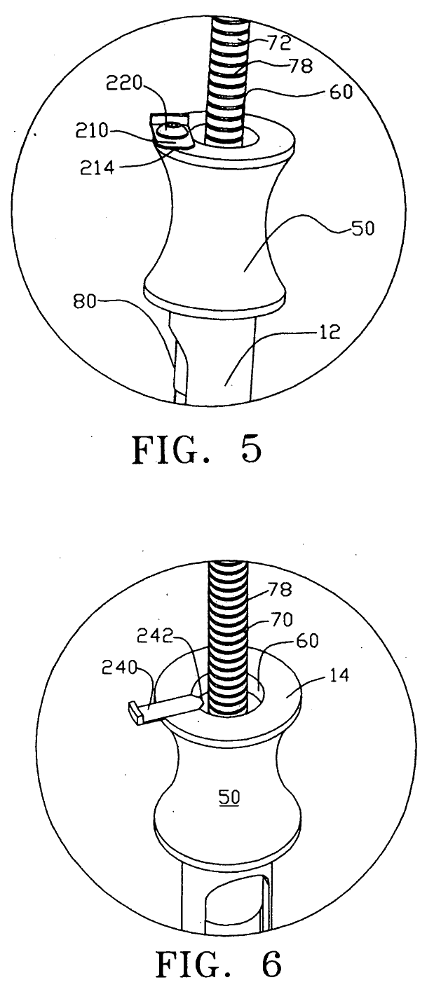

Turning now to FIGS. 4-5 in which a dental syringe 200 according to a second embodiment is illustrated. The dental syringe 200 is similar to syringe 10 of FIGS. 1-3 and therefore, like elements are numbered alike.

The main difference between the dental syringe 10 and the dental syringe 200 is with the dosage dispensing feature and more specifically, the dental syringe 200 has a different type of dosage dispensing feature 210 for controllably dispensing a predetermined amount of medication as the plunger 70 is incrementally advanced within the body 12. In this second embodiment, the dosage dispensing feature 210 is selectively placed in an active position where it can engage the plunger 70 as shown in FIG. 4 or the dosage dispensing feature 210 can be placed into an inactive, disengaged position where it is offset from the plunger 760 and is prevented from engaging the plunger 70.

Similar to the dosage dispensing feature 100, the dosage dispensing feature 210 is in the form of an elongated flange 210 that has a first end 212 and an opposing second end 214. Unlike the flange 110, the flange 210 is not fixedly attached to the body 12 but instead the first end 212 is pivotably attached to the body 12 so as to permit the flange 210 to be moved into the active, engaged position shown in FIG. 4 or to be moved into the inactive, disengaged position shown in FIG. 5.

The illustrated flange 210 includes a lip 216 at the first end 212 to assist the user in pivoting the flange 210 between the engaged and disengaged positions. The lip 216 protrudes upwardly from the top surface of the flange 210 such as at a right angle. The flange 210 can be pivotably attached to the body 12 at the proximal end 14 using a coupling member 220. The coupling member 220 can be any number of different types of structures, such as a fastening member. For example, the coupling member 220 can be a screw or rivet or the like that permits the flange 220 to pivot as shown. In the case of where the coupling member 220 is a screw or the like, the user loosens the screw to permit the flange 220 to pivot into and out of engagement with the plunger 70. To lock the flange 220 into the desired position (the engaged position or disengaged position), the user simply tightens the coupling member 220.

While the coupling member 220 is illustrated as being a conventional screw with a recessed head section to receive a tool for tightening or loosening the coupling member 220, it will be appreciated that the head of the coupling member 220 can instead contain a projection or grip portion that can be gripped by the user and permits the user to easily loosen or tighten the coupling member instead of using a tool. For example, a small tab can extend up from the head of the coupling member and can be gripped by the user to permit rotation of the coupling direction in either a tightening direction or a loosening direction.

In yet another embodiment illustrated in FIGS. 6-8, a top surface of the finger holder 50 has a guide track or recessed guide channel 230 formed therein and extends from an outer peripheral edge 232 of the finger holder 50 to an inner peripheral edge 234 that defines the opening 60 at the proximal end 14 of the body 12. The guide channel 230 thus provides an entrance into the opening 60.

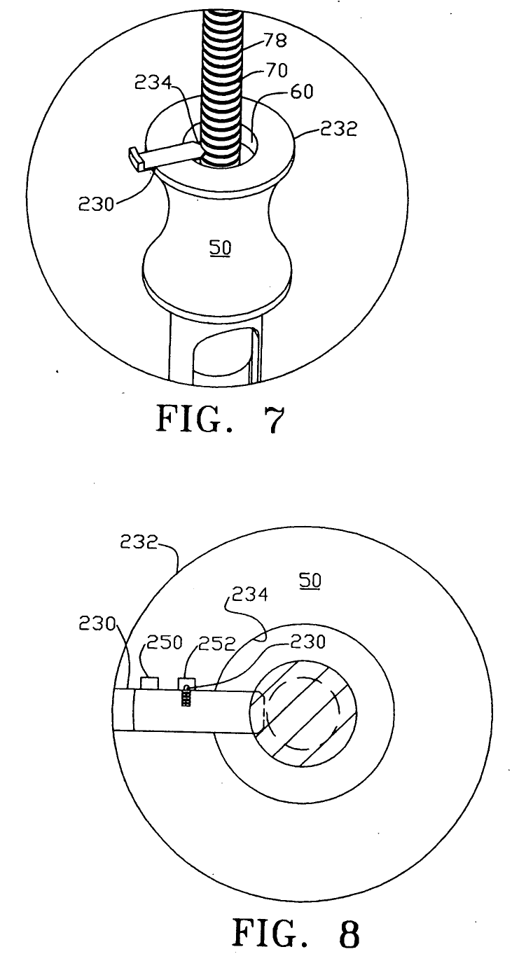

In this embodiment, the dosage dispensing feature is in the form of a slideable flange 240 that travels within the guide channel 230 between a disengaged position shown in FIG. 6 and an engaged position shown in FIG. 7. The guide channel 230 has a generally rectangular shape that is complementary to the flange 240 so that the flange 240 can be received within and can slideably travel within the guide channel 230 such that one end 242 of the flange 240 extends into the opening 60 and into engagement with the channel 78 formed in the plunger rod 12.

The guide channel 230 can have two locating and locking features 250, 252 formed as a part thereof that permit the flange 240 to be locked in a first position in which the flange 240 is disengaged from the plunger 70 and a second position in which the flange 240 engages the plunger 70. The first locking feature 250 is in the form of a detent that extends outwardly from and is in communication with the guide channel 230 and is formed in the top surface of the feature 50 closer to the outer peripheral edge 232. The second locking feature 250 is in the form of a detent that extends outwardly from and is in communication with the guide channel 230 and is formed in the top surface of the feature 50 closer to the inner peripheral edge 234. The detents 250, 252 can have any number of different shapes and sizes so long as they are complementary to the shape of the flange 240.

Unlike the other embodiments, the flange 240 has a first tab 260 that is complementary to each of the detents 250, 252. Preferably the first tab 260 has rounded surfaces or edges to more easily permit the disengagement of the first tab 260 from one detent 250, 252.

It will therefore be appreciated that when the flange 240 is in the first position, where it is disengaged and spaced from the plunger 70, the first tab 260 is securely received within the detent 250 so as to lock the flange 240 in place. When the flange 240 is in the second position, where it engages the plunger 70, the first tab 260 is securely received within the detent 252 so as to lock the flange 240 in place with the plunger 70 such that the end 242 of the flange 240 is received in one channel or groove 78 of the plunger rod 72.

According to one embodiment, the first tab 260 is freely flexible so that when the user wishes to move the flange 240 between the first and second positions, the axial movement of the flange 240 in the guide channel 230 in either direction causes the first tab 260 to flex out of engagement with one of the detents 250, 252 until the flange 240 is moved into the other position where the first tab 260 flexes back into engagement with the other detent 250, 252. For example, the material that forms the first tab 260 can be formed of a synthetic material, such as a foam material, so long as once the first tab 260 flexes, the first tab 260 and the flange 240 can travel within the guide channel 230 until the first tab 260 is aligned with the other detent 250, 252. As soon as the first tab 260 is aligned with the other detent 250, 252, the memory characteristics of the first tab 260 cause the first tab 260 to flex outwardly into engagement with the other detent 250, 252.

In yet another embodiment and shown in FIG. 8, the first tab 260 is biasedly coupled to the flange 240 so that when the first tab 260 is aligned with one of the detents 250, 252, the first tab 260 is spring biased into the detent 250, 252. Conversely when the flange 240 is moved longitudinally within the guide channel 230, the first tab 260 is biased inward toward to the flange 240 and out of engagement with the respective detent 250, 252 to permit the flange 240 to move within the guide channel 230. Once the first tab 260 is aligned with the detent 250, 252, the spring biased characteristics of the first tab 260 causes the first tab 260 to be spring biased outwardly away from the flange 240 and into engagement with the detent 250, 252 to cause the flange 240 to be releasably locked in one of the first and second positions, respectively.

Any number of different ways for spring biasing the first tab 260 relative to the flange 240 can be employed. For example, the body of the flange 240 can include a compartment formed therein that is open along one side thereof. An inner end of the first tab 260 that is contained within the compartment has a lip or the like that contacts and is restricted by the side wall of the flange 240 so that the outward movement of the first tab 260 is limited due to the contact between the lip and the side wall. The lip thus acts as a stop for the flange 240; however, when the lip is in contact with the side wall, the first tab is in a fully extended position and is received within one of the detents 250, 252. Between a floor of the side compartment and the inner end of the flange, a biasing member, such as a spring, 270 is provided for applying an outward biasing force against the flange 240. Thus, in a normal, rest position, the flange 240 is biased outwardly away from the body of the flange 240 to permit reception into one of detents 250, 252. To move the flange 240 within the guide channel 230, a force must be applied that overcomes the biasing force and causes retraction of the flange 240 within the compartment, and thus, permit longitudinal movement of the flange 240 within the guide channel 230.

It will be appreciated that in this embodiment, the user does not have to tighten or loosen a coupling member that attaches the flange to the body.

It will be understood that the dental syringes according to the present invention each includes a flexible element (flange) coupled to the body of the syringe and being configured to engage a complementary feature (groove) formed along the longitudinal length of the plunger rod. When the syringe plunger is pushed down through the body, the grooves on the plunger pass the flexible element and produce an audible “click” feedback and/or tactile feedback through the finger pushing the plunger. Because the grooves or channels formed along the plunger rod are placed at precise uniform distances (e.g., every 1/10^{th} of the medication cartridge) the dentist is much better aware of the amount of medication he or she is injecting into the patient through the auditory/tactile feedback.

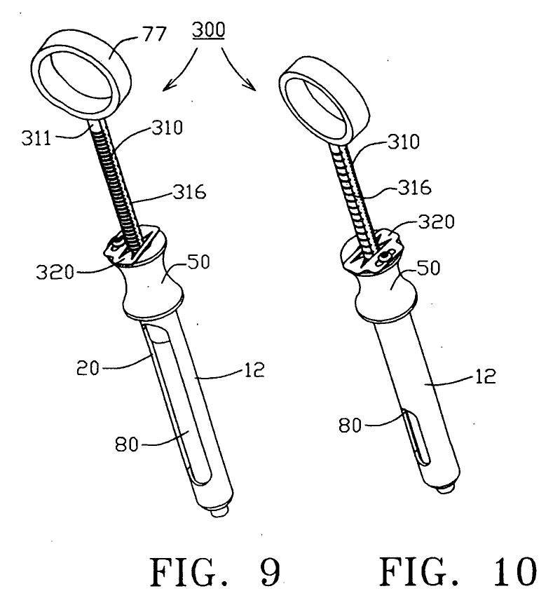

FIGS. 9-13 illustrate another embodiment of the present invention. In this embodiment, a dental syringe 300 includes the body 12 and cartridge 80; however, a different type of plunger 310 is provided that mates with a dosage dispensing feature 320 that is associated with the body 12 and in particular, is coupled to the top surface of the finger holder 50.

In this embodiment, the plunger 310 is similar to the plunger 70 in that it contains plunger rod 311 with thumbring 78 and the stopper/barb at the other end. However and in accordance with the present invention, the plunger 310 is constructed so that it includes a first set of spaced grooves 312 formed longitudinally along one section/length of the plunger 310 and a second set of spaces grooves 314 formed longitudinally along a different section/length of the plunger 310, with a distance between the first set of grooves 312 being different than the distance between the second set of grooves 314 as illustrated in FIGS. 9-13. In the illustrated embodiment, the distance A between the first set of spaced grooves 312 is greater than the distance B between the second set of spaced groves 314. It will therefore be appreciated that each of the grooves 312, 314 does not extend completely around the circumference of the plunger rod 311 but instead each groove 312, 314 only extends along a length or portion of the plunger rod 311. For example when there are two sets of different grooves or channels 312, 314, each set of grooves 312, 314 occupies less than one half the area of the plunger rod 311. As described in more detail below, when there are more than two sets of grooves formed longitudinally along the plunger rod 311, each set of grooves occupies less than one half of the area of the plunger rod 311.

The illustrated plunger rod 311 also contains a pair of flats 316 formed longitudinally along the length of the plunger rod 311. Each flat 316 is a planar surface that is formed longitudinally along the length of the plunger rod 311 and when there are two flats 316, the two planar surfaces 316 are parallel to one another. While a portion of the grooves 312, 314 can extend and be formed partially within the flats 316, the majority of the grooves 312, 314 is formed in the arcuate sections of the plunger rod 311 that are formed between the flats 316. In combination with the dosage dispensing feature 320, the flats 316 prevent rotation of the plunger rod 311 within the plunger body 12.

It will be understood that the precise distances between the grooves 312 and the grooves 314 can be varied depending upon the precise applications and dispensing needs. The spacing of the first set of grooves 312 provides dispensing of a first dosage amount, while the spacing of the second set of grooves 314 provides dispensing of a second dosage amount. Once again, further incremental movement of the plunger within the body results in successive dosage amounts being dispensed when the dosage dispensing feature 320 engages either grooves 312 or grooves 314.

FIGS. 9-13 illustrate one dosage dispensing feature 320 according to an exemplary embodiment. The dosage dispensing feature 320 is in the form of a plate 322 that is coupled to the top surface of the finger holder 50. The plate 322 has a first opening 324 formed therethrough and a second opening 326 formed therethrough. The first opening 324 is constructed so that a pair of opposing flexible teeth or flanges 330, 332 are formed. More specifically, the first opening 324 is formed so that the two flexible flanges 330, 332 are formed near the ends of the first opening 324, with the first opening 324 also being defined by a pair of parallel side edges 336 that define, in part, the two flanges 330, 332. Each of the illustrated flexible flanges 330, 332 is in the form of a substantially pointed, triangular shaped member that is capable of engaging one of the groves 312, 314. The two free ends of the flexible flanges 330, 332 are axially aligned with one another.

The second opening 326 is in the form of a slot (e.g., oval or oblong shaped) that is spaced from the first opening 324. The second opening 326 can receive a fastener 340 or the like that permits the plate 322 to be positioned on and coupled to the top surface of the finger feature 50. As described below, by loosening the fastener 340, the plate 322 can be moved along the top surface and repositioned on the top surface to permit the flexible flange 330 to engage one groove 312 or permit the flexible flange 332 to engage one groove 314. Accordingly, only one of the flexible flanges 330, 332 engages one of the sets of grooves 312, 314, respectively, at any one time and it is not possible for both flanges 330, 332 to simultaneously engage the grooves 312, 314.

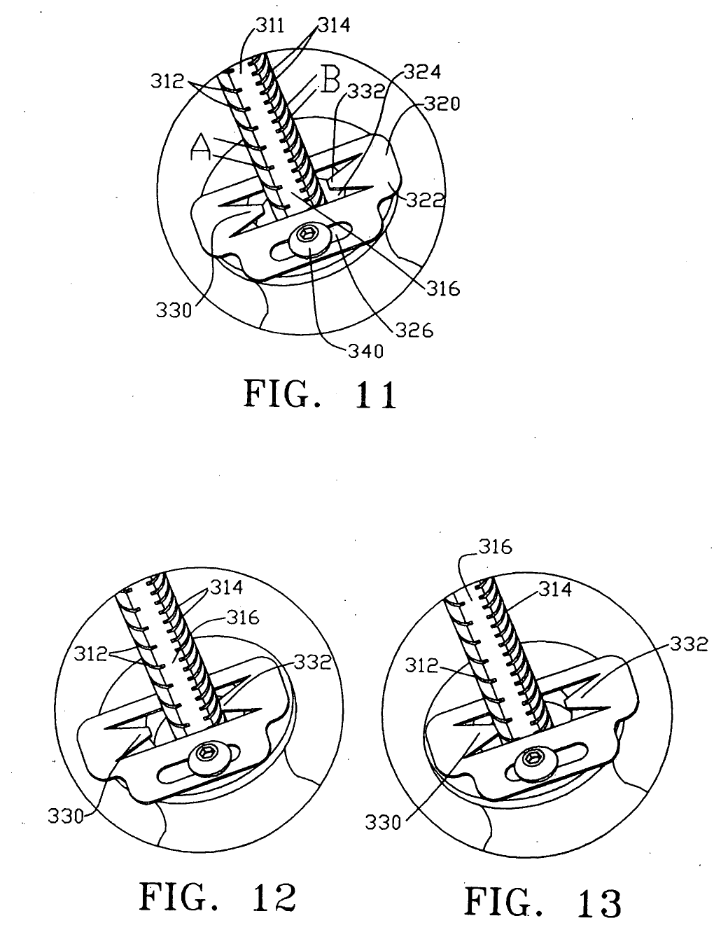

Accordingly, based on the position of the plate 322 relative to the top surface of the feature 50, the flexible flanges 330, 332 can be placed in any of three different positions. A first position shown in FIG. 11 is where neither of the flanges 330, 332 are in engagement with the plunger 310; a second position shown in FIG. 12 is where only the flexible flange 332 engages one groove 314 of the plunger 311 and a third position shown in FIG. 13 is where only the flexible flange 330 engages one groove 312 of the plunger 311. The relative position of the fastener 340 within the second opening (slot) 326 also varies according to whether the dosage dispensing feature 320 is in the first, second or third position and in particular, in the first position, the fastener 340 is in the center of the slot 326, in the second position, the fastener 340 is located in the left end of the slot 326, and in the third position, the fastener 340 is located in the right end of the slot 326.

It will be appreciated that the dental syringe 300 offers two different modes of dispensing medication in that when the flange 330 engages the set of grooves 312, medication can be incrementally dispensed in doses having a first predetermined volume, while, when the flange 332 engages the set of grooves 314, medication can be incrementally dispensed in doses having a second predetermined volume that is different from the first predetermined volume. The flats 316 and parallel side edges of the plate 312 prevent the rotation of the plunger 310 and therefore ensure that the flange engages the desired set of grooves.

It will also be appreciated that in another embodiment, there is only one flange 332 that engages either the first set of grooves 312 or the second set of grooves 314 depending on the position of the plunger 310 within the body 12, i.e., whether the single flange 332 is facing the grooves 312 or the grooves 314. In other words, the plunger 310 can be of the type that can be removed and reinserted so that a different set of grooves is facing the one flange 332 for engagement therewith.

It will be understood that while the use of a plunger with two or more distinct gradation arrangements in the form of two separate sets of grooves that are each spaced from one another by different distances has been described with reference to a dental syringe, this type of plunger construction can be used with other types of syringes, including conventional medical syringes (hypodermic syringes).

The plunger can also include a keying feature which in combination with a complementary keying feature associated with the syringe body permits the plunger to be inserted into the syringe body 12 in only one orientation to ensure the proper mating with the dosage dispensing feature and the plunger. For example, the opening 60 at the proximal end 14 of the barrel 12 that is formed through the finger holder 50 can have a keying notch or the like that extends radially outward therefrom and forms an entrance into a locating channel that extends longitudinally within the body 12. The plunger 70 has a tab, projection or the like that extends outwardly therefrom and is sized to fit within the notch and into the locating channel where it travels longitudinally as the plunger is advanced either toward or away from the body 12. Since the locating channel is kept separate from the bore or interior 18 that receives the plunger rod 72, the plunger 70 is prevented from freely rotating within the interior 18 due to the projection being captured within the locating channel. In this manner and when the plunger contains two sets of differently spaced grooves, there are two locating channels formed in the body such that when it is desired for the dosage dispensing feature (flexible flange) to engage one set of grooves of the plunger, the projection is placed in one of the locating channels and when it is desired for the dosage dispensing feature to engage the other set of grooves of the plunger, the projection is placed in the other locating channel.

Once again, it will be understood that the dosage dispensing features described herein, including the use of a plunger with two different set of tactile dispensing features, are not limited to being used with a dental syringe but instead can be used with other syringes, such as standard hypodermic syringes that draw in and discharge fluids, etc.

While the dental syringe of the invention has been described to be adapted for dispensing a medication, such as a Novocain, it may be applicable to other systems. Furthermore, application of the present device can be successfully utilized in various industries requiring a metered distribution of fluid or other matter. Thus the foregoing description and accompanying drawings set forth the preferred embodiment of the invention. Modifications, alternative designs will be apparent in light of the foregoing teaching without departing from the scope of the appended claims.

What is claimed is:

1. A dental syringe comprising:
a barrel extending along a longitudinal axis and including an interior space and a longitudinal window formed in a side wall of the barrel for receiving a cartridge that contains medication; a flexible flange member formed at a proximal end of the barrel with one end of the flexible flange member extending into an opening at the proximal end that forms an entrance into the interior space of the barrel; and
a plunger received in and axially displaceable within the interior space; and a plurality of spaced grooves provided on an outer surface of the plunger, wherein the flange is configured to selectively engage the grooves while generating at least one indicating signal corresponding to a predetermined dosage of medication dispensed from the cartridge during axial displacement of the plunger as the flange engages at least one groove.

2. The dental device of claim 1, wherein the flange is formed of flexible material selected from the group consisting of a plastic and a metal and the barrel and plunger are formed of metal.

3. The dental syringe of claim 1, wherein the barrel has a contoured finger holder formed at a proximal end thereof, the finger holder having an upper surface to which the flange is attached.

4. The dental syringe of claim 3, wherein the flange is pivotally attached to the upper surface such that is can be moved between a first position where one end of the flange engages the groove of the plunger and a second position where the one end of the flange is spaced from and disengaged from the groove of the plunger.

5. The dental syringe of claim 1, wherein the barrel has a contoured finger holder formed at a proximal end thereof, the finger holder having an upper surface with a recessed guide track formed therein, the flange being slideably contained within the guide track and being moveable between a first position where one end of the flange engages the groove of the plunger and a second position where the one end of the flange is spaced from and disengaged from the plunger.

6. The dental syringe of claim 5, wherein the guide track has a first stop and a second stop that communicate and extend outwardly from the guide track, the flange including a tab extending outwardly therefrom such that when the tab is received in the first stop, the flange is locked in the first position and when the tab is received in the second stop, the flange is locked in the second position.

7. The dental syringe of claim 6, wherein the tab is formed of a flexible material to permit flexing of the tab to permit disengagement of the tab from one of the first and second stops and longitudinal movement of the flange within the guide channel.

8. The dental syringe of claim 6, wherein the first and second stops are formed at right angles to the guide track.

9. The dental syringe of claim 6, wherein the tab is spring biased relative to the flange such that in an unbiased condition, a biasing element extends the tab into one of the first and second stops and in a biased condition, the biasing element stores energy to permit retraction of the tab within the flange to permit longitudinal movement of the flange within the guide channel.

10. The dental syringe of claim 9, wherein the biasing element is a spring that applies a force to an inner end of the tab, the inner end having an oversized lip that acts as a stop and limits the movement of the tab outwardly from the flange when the lip contacts a side wall of the flange.

11. The dental syringe of claim 1, wherein the flange is formed of a material selected from the group consisting of a plastic and a metal and the barrel and plunger are formed of metal.

12. The dental syringe of claim 1, wherein the grooves extend around a complete circumference of the plunger, the grooves being spaced a predetermined distance from one another, with a distance between two adjacent grooves being equal to a single dosage of medication.

13. The dental syringe of claim 1, wherein grooves extend only partially around a circumference of the plunger, the grooves being spaced a predetermined distance from one another, with a distance between two adjacent grooves being equal to a single dosage of medication.

14. The dental syringe of claim 4, wherein the flange is pivotally attached to the finger holder with a coupling member that can be loosed to permit pivoting of the flange between the first and second positions and tightened to permit locking of the flange in one of the first and second positions.

15. A dental syringe comprising:
a barrel extending along a longitudinal axis and including an interior space and a longitudinal window formed in a side wall of the barrel for receiving a cartridge that contains medication;
a flexible flange member formed at a proximal end of the barrel with one end of the flexible flange member extending into an opening at the proximal end that forms an entrance into the interior space of the barrel; and
a plunger received in and axially displaceable within the interior space; the plunger having a first set of grooves formed therein along a longitudinal length of the plunger and spaced a first distance apart from one another and a second set of grooves formed therein along the longitudinal length of the plunger and spaced a second distance from one another that is different from the first distance, wherein the flexible flange member is configured to selectively engage one of the first set of grooves and the second set of grooves while generating an indicating signal corresponding to a predetermined dosage of medication dispensed from the cartridge during axial displacement of the plunger as the flexible flange member engages successive grooves.

16. The dental syringe of claim 15, wherein the first set of grooves occupies a first half the plunger and the second set of grooves occupies the other half of the plunger.

17. The dental syringe of claim 15, wherein the plunger includes a pair of flats formed longitudinally along a length of the plunger with the first and second set of grooves being formed along arcuate end sections of the plunger.

18. The dental syringe of claim 17, wherein a portion of one of the first and second sets of grooves extends into the pair of flats.

19. The dental syringe of claim 17, wherein the pair of flats are formed parallel to one another and face complementary planar, parallel surfaces of the flexible flange member to prevent rotation of the plunger in the interior.

20. The dental syringe of claim 15, wherein the flexible flange member comprises a plate that is moveably coupled to the proximal end of the barrel and includes a first cutout formed therein for receiving the plunger, the cutout defining a first flexible flange and a second flexible flange, the first flange corresponding to the first set of grooves, while the second flange corresponding to the second set of grooves, with the plunger being received between the first and second flanges.

21. The dental syringe of claim 20, wherein the plate contains a slot formed therethrough to receive a coupling member for attaching the plate to the proximal end, wherein one of the first and second flanges is moved into engagement with one of the first and second set of grooves by moving the plate relative to the coupling member and then tightening the coupling member.

22. The dental syringe of claim 15, wherein the plunger has a third set of grooves formed therein along a longitudinal length of the plunger and spaced a third distance apart from one another, wherein the third distance is different from each of the first and second distances, the first, second and third set of grooves being formed equidistant from one another.

23. A dental syringe comprising:
a barrel extending along a longitudinal axis and including an interior space and a longitudinal window formed in a side wall of the barrel for receiving a cartridge that contains medication;
a dosage dispensing member associated with the barrel for administering a predetermined dosage amount of medication; and
a plunger received in and axially displaceable within the interior space; the plunger having first and second dosage dispensing features formed as a part thereof for selectively permitting two different dosage amounts of medication to be dispensed based on the orientation of the plunger within the barrel and based on which of the first and second dosage dispensing features is operatively coupled to the dosage dispensing member.

24. A method of controllably and selectively evacuating or dispensing a predetermined dosage of medication comprising the steps of:
providing a dental syringe including:
a barrel extending along a longitudinal axis and including an interior space and a longitudinal window formed in a side wall of the barrel for receiving a cartridge that contains medication; a flexible flange member formed at a proximal end of the barrel with one end of the flexible flange member extending into an opening at the proximal end that forms an entrance into the interior space of the barrel; and
a plunger received in and axially displaceable within the interior space; and a plurality of spaced grooves provided on an outer surface of the plunger, wherein the flange is configured to selectively engage the grooves while generating at least one indicating signal corresponding to a predetermined dosage of medication dispensed from the cartridge during axial displacement of the plunger as the flange engages successive grooves; and
axially displacing a plunger within the interior space such that the flange engages at least one other groove while generating an indicating signal corresponding to the predetermined dosage of medication dispensed from the cartridge during axial displacement of the plunger.
